(19) United States
(12) Patent Application Publication
(10) Pub. No.: US 2025/0049693 A1
(43) Pub. Date: Feb. 13, 2025

(54) METHOD FOR AMPLIFYING EXPRESSION OF TRANSPORTER-RELATED GENE IN EPIDERMAL CELLS

(71) Applicant: J-Network, Inc., Newport Beach, CA (US)

(72) Inventors: Tatsuro MIYOSHI, Newport Beach, CA (US); Brian Charles KELLER, Newport Beach, CA (US); Akira KODAMA, Newport Beach, CA (US)

(21) Appl. No.: 18/744,338

(22) Filed: Jun. 14, 2024

(30) Foreign Application Priority Data
Aug. 9, 2023 (JP) 2023-129694

(51) Int. Cl. A61K 8/86; A61K 8/34; A61Q 19/00

(52) U.S. Cl. CPC A61K 8/86; A61K 8/347; A61Q 19/00

(57) ABSTRACT

An action of amplifying expression of transporter-related genes in epidermal cells by a diacylglycerol PEG adduct is utilized. A method for amplifying expression of transporter-related genes in epidermal cells is provided. The method comprises applying a diacylglycerol PEG adduct, wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

### FIELD

The preset invention relates to a method for amplifying expression of a transporter-related gene in epidermal cells.

### BACKGROUND

Japanese Patent No. 4497765 (Self-forming, thermodynamically stable liposomes and their applications) discloses a preparation method in which a diacylglycerol polyethylene glycol adduct (hereinafter referred to as “diacylglycerol PEG adduct”) is utilized as a lipid molecule and mixed with water or a surfactant to spontaneously form a vesicle. Such a vesicle is used in a drug delivery system in which a target substance such as a protein or a drug is encapsulated in or bound to the inside or surface of the vesicle and delivered to cells in a living body. A vesicle composed of a diacylglycerol PEG adduct has a form in which the surface thereof is covered with a hydrophilic PEG chain, and has good permeability into a living body and good stability in blood.

Japanese Patent No. 6297737 (Preparing method for positively-electrified charged noisome, and charged niosome) describes that a charged element is bound to the surface of a vesicle composed of a diacylglycerol PEG adduct and positively-electrified, thereby improving permeability and retention of the vesicle in the stratum corneum of the epidermis.

The vesicle in a drug delivery system has been recognized simply as a carrier of a drug which is a target substance. In recent years, it has been known that the molecule itself of the diacylglycerol PEG adduct derived from the vesicle degraded in the living body also exerts a useful action in the living body.

Japanese Patent No. 6805385 (Expression enhancer of moisturizing related substances in epidermis) discloses that a diacylglycerol PEG adduct contributes to enhancement of expression of moisturizing related substances such as profilaggrin, filaggrin, and natural moisturizing factor NMF in the epidermis. In addition, Japanese Patent No. 6860739 (Expression enhancer of antioxidant in epidermis) discloses that a diacylglycerol PEG adduct contributes to enhancement of expression of antioxidant-related substances such as Nrf2 and PPARG, which are oxidative stress-response genes, as well as NQO-1, CAT, and HMOX1, which are antioxidant enzymes, in the epidermis.

Meanwhile, various transport proteins (so-called transporters) present in cell membranes function in the incorporation and excretion of various substances into and from epidermal cells. For example, a sodium-dependent vitamin C transporter (SVCT) that transports vitamin C into cells has been found, and type 1 (SVCT1) and type 2 (SVCT2) are known (WO 2007/094312 (Production enhancer of vitamin C transporter)).

Further, for example, an xCT protein related to the xCT mechanism in which cystine, which is one of amino acids, is incorporated into cells and glutamic acid in the cells is excreted to the outside of the cells is known. The cystine incorporated into the cells is involved in the production of glutathione via cysteine (Japanese Patent Application Laid-open No. 2010-280675 (Compositions for potentiating glutathione)).

In addition, for example, an ABCC1 protein, which is one of ABC-transporters having an action of excreting various substances from the inside to the outside of the cells, is known (Japanese Patent Application National Publication No. 2022-514669 (Composition comprising Lactobacillus Rhamnosus extract)).

In addition, for example, an aquaporin protein, which functions as a water channel that regulates the amount of moisture in the cells, is known, and in particular, aquaporin 3 (AQP3) is abundant in the epidermis (Japanese Patent Application Laid-open No. 2011-32191 (Aquaporin 3 expression regulator)).

As described above, various transporters in epidermal cells are known, but there are few reports of substances that enhance the expression of these transport proteins. Also with respect to diacylglycerol PEG adducts, how they relate to various mechanisms of incorporation or excretion of drugs or the like into or from epidermal cells is little known.

An object of the present invention is to utilize a newly found property associated with transporters in epidermal cells in diacylglycerol PEG adducts, and in particular to utilize a property that amplifies the expression of transporter-related genes in epidermal cells.

### SUMMARY

In order to achieve the above object, the present invention provides the following configurations.

An aspect of the present invention provides a method for amplifying expression of a transporter-related gene in epidermal cells, the method comprising applying a diacylglycerol PEG adduct having a following structural formula;
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

Another aspect of the present invention provides a method for using an active ingredient for amplifying expression of a transporter-related gene in epidermal cells in a cosmetic or a skin external preparation, the method comprising using, as the active ingredient, a diacylglycerol PEG adduct having a following structural formula;
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

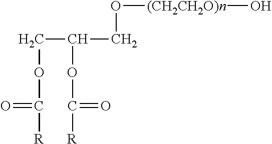

It is preferred that the transporter-related gene in epidermal cells is at least one of a group consisting of SVCT2, SLC7A11, glutathione reductase, ABCC1, and aquaporin 3.

It is preferred that the diacylglycerol PEG adduct permeates into epidermis in a solution state or a vesicle state.

The cosmetic or the skin external preparation can further comprise at least one of a group consisting of ascorbic acid, an ascorbic acid derivative, glutathione, and cysteine, and in that case, the cosmetic or the skin external preparation can further comprise hydroquinone.

According to the present invention, amplification of expression of transporter-related genes in epidermal cells is realized by diacylglycerol PEG adducts.

Further, according to the present invention, amplification of expression of transporter-related genes in epidermal cells is realized by a cosmetic or a skin external preparation containing a diacylglycerol PEG adduct as an active ingredient.

### BRIEF DESCRIPTION OF DRAWINGS

FIG. 1 is a graph illustrating relative expression levels of SVCT2 genes in GDS23;

FIG. 2 is a graph illustrating relative expression levels of SVCT2 genes in GDM12;

FIG. 3 is a graph illustrating relative expression levels of SLC7A11 genes in GDS23;

FIG. 4 is a graph illustrating relative expression levels of SLC7A11 genes in GDM12;

FIG. 5 is a graph illustrating relative expression levels of GSR genes in GDS23;

FIG. 6 is a graph illustrating relative expression levels of GSR genes in GDM12;

FIG. 7 is a graph illustrating relative expression levels of ABCC1 genes in GDS23;

FIG. 8 is a graph illustrating relative expression levels of ABCC1 genes in GDM12;

FIG. 9 is a graph illustrating relative expression levels of AQP3 genes in GDS23;

FIG. 10 is a graph illustrating relative expression levels of AQP3 genes in GDM12;

FIG. 11 is a band image of Western blot of a SVCT2 protein with respect to GDS23;

FIG. 12 is a graph illustrating expression levels of the SVCT2 protein;

FIG. 13 is a graph illustrating evaluation test results of a cellular uptake action of vitamin C; and

FIG. 14 is a graph illustrating evaluation test results of a cellular uptake action of cystine with respect to GDS23.

### DESCRIPTION OF EMBODIMENTS

Embodiments of the present invention will be described below with reference to the drawings.

The present invention has been made by utilizing a newly found property in a diacylglycerol polyethylene glycol adduct (diacylglycerol PEG adduct). The newly found property is the action of amplifying the expression of transporter-related genes in human epidermal cells and genes related thereto.

The structural formula of the diacylglycerol PEG adduct which is a lipid molecule according to the present invention is schematically illustrated.

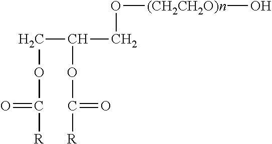

Diacylglycerol PEG adducts are composed of a glycerol skeleton (CH₂CHCH₂) having three carbons, a PEG chain which is a linear polyethylene glycol bonded to one carbon at the terminal of the three carbons of the skeleton, and the same type of long chain fatty acid (COOR) bonded to each of the other two of the three carbons. Part of the PEG-chain is hydrophilic and part of the long chain fatty acid is hydrophobic.

When a specific diacylglycerol PEG adduct is described in the following description, it is referred to as “[PEG-n]+[glycerol]+[di]+[long chain fatty acid name]” based on the type of the long chain fatty acid and the number n of the PEG chain. For example, when the long chain fatty acid is myristic acid and n of the PEG chain is 12, it is “PEG-12 glycerol dimyristate”. In addition, a specific diacylglycerol PEG adduct may be further abbreviated.

The number of carbon atoms of R in the long-chain fatty acid can be in a range of 11 to 23. The long-chain fatty acids included in this range are, for example, myristic acid, palmitic acid, stearic acid, oleic acid or the like. The number n of PEG chains can be in the range of 11 to 46. Examples of the diacylglycerol PEG adducts related to the present invention include the following substances. Melting points and abbreviations are indicated in parentheses.
PEG-12 glycerol dimyristate (25.0° C.: GDM12)
PEG-12 glycerol distearate (40.0° C.: GDS12)
PEG-23 glycerol distearate (39.8° C.: GDS23)
PEG-23 glycerol dipalmitate (31.2° C.: GDP23)
PEG-12 glycerol dioleate (25.0° C.: GDO12)

Various transport proteins (so-called transporters) present in the cell membrane of human epidermal cells are responsible for incorporation and excretion of various substances into and from epidermal cells. SVCT2, SLC7A11, GSR, ABCC1, and aquaporin 3 are transporters in epidermal cells and genes related thereto, the expression of which has been confirmed to be amplified by diacylglycerol PEG adducts in the present invention. These are collectively referred to herein as “transporter-related genes in epidermal cells”. Each of these genes encodes a corresponding protein or enzyme.

The SVCT2 protein encoded by the SVCT (sodium-dependent vitamin C transporter) 2 gene forms a pathway necessary for vitamin C (ascorbic acid) to pass into cells. Vitamin C has an action of inhibiting melanin production in epidermal cells and contributing to whitening.

Ascorbic acid can be administered to the skin, for example, as an ingredient of a cosmetic or the like. Since ascorbic acid is insufficient in stability and skin permeability, ascorbic acid derivatives improved in these properties are also used in place of ascorbic acid or in combination with ascorbic acid. Ascorbic acid derivatives are converted into ascorbic acid in the skin or act equivalently to ascorbic acid.

The xCT (cystine/glutamate transporter) protein encoded by the SLC7A11 gene incorporates cystine, which is one of amino acids, into cells. The cystine incorporated into the cells is reduced to cysteine, and the cysteine produces glutathione (reduced glutathione), which is a tripeptide, together with glutamic acid and glycine. Reduced glutathione is one of antioxidants that protect cells from oxidation by itself being oxidized (oxidized glutathione).

Cystine can be synthesized in the body, but cysteine can also be administered to the skin, for example, as an ingredient of cosmetics or the like. Exogenously administered cysteine is converted to dimeric cystine in the skin and is incorporated into cells by the xCT transporter.

Glutathione reductase encoded by the GSR (glutathione-disulfide reductase) gene is an enzyme that reduces oxidized glutathione back to reduced glutathione. Although GSR is not a transporter, it is considered to be transporter-related genes in the present invention because it is an enzyme that acts in conjunction with the xCT and ABCC1 transporters.

Glutathione is an amino acid that can be synthesized in the body, but it can be administered to the skin, for example, as an ingredient of cosmetics or the like. Exogenously administered glutathione (reduced glutathione) is also reduced by glutathione reductase when oxidized in the skin.

The ABCC1 protein, which is encoded by the ABC (ATP binding cassette) CI gene, is a member of the ABC transporter family of proteins and is also referred to as MRP1. The ABCC1 protein excretes glutathione, to which toxic substances such as intracellular toxins are attached, to the outside of the cell.

The aquaporin 3 protein encoded by the aquaporin 3 gene functions as a water channel that regulates the amount of moisture in cells in the epidermis. The aquaporin 3 protein is responsible for moisturizing and maintaining elasticity of the skin by allowing water to spread throughout the epidermis.

The present inventors have found that the expression of transporter-related genes in epidermal cells is amplified by applying a diacylglycerol PEG adduct to human epidermis. By amplifying the expression of transporter-related genes in epidermal cells, the production of proteins and enzymes encoded by these genes is enhanced. This is a novel action of the diacylglycerol PEG adduct relating to membrane transport of the human epidermis, particularly the epidermal cell membrane, and is a novel property of the diacylglycerol PEG adduct. This property can provide effects such as whitening, antioxidation, detoxification, moisturizing, and the like to the epidermis. This is not simply a physical protective effect on the epidermal surface, but an effect obtained in epidermal cells.

The present invention provides a method for amplifying the expression of transporter-related genes in epidermal cells by applying a diacylglycerol PEG adduct utilizing the newly found property of the diacylglycerol PEG adduct.

The present invention also provides a method for using the diacylglycerol PEG adduct in a cosmetic or a skin external preparation as an active ingredient for amplifying the expression of transporter-related genes in epidermal cells.

In the present invention, when the diacylglycerol PEG adduct is applied to the human epidermis, only one kind of the diacylglycerol PEG adduct may be used, or a plurality of kinds thereof may be used in combination.

According to the present invention, the diacylglycerol PEG adduct that has reached the inside of the epidermis can amplify the expression of transporter-related genes in epidermal cells and increase the production amount of proteins and enzymes corresponding thereto, as compared with the case where the diacylglycerol PEG adduct is not present. As a result, the state of not only the inside of the epidermis but also the surface of the epidermis is improved. By carrying out the method of the present invention, it is possible to provide a cosmetic or a skin external preparation containing, as an active ingredient, a diacylglycerol PEG adduct as an expression amplifier for transporter-related genes in epidermal cells. Both the cosmetic and the skin external preparation are applied to the epidermal surface. Here, those other than the cosmetic are generally referred to as skin external preparations, and examples thereof include ointments or the like. Such a cosmetic or a skin external preparation can be provided in various forms such an aqueous solution, a milky lotion, a gel, a cream, and the like. These cosmetics or skin external preparations can contain, in addition to the diacylglycerol PEG adduct, other active ingredients and/or various ingredients that are generally contained.

As one method for allowing the diacylglycerol PEG adduct to reach the human epidermis, the diacylglycerol PEG adduct can be allowed to reach the epidermis in a state of a solution prepared by dissolving the diacylglycerol PEG adduct in water or a predetermined solvent. For example, a solution of the diacylglycerol PEG adduct having a predetermined concentration is prepared by using a phosphate-buffered saline solution PBS (−) as a solvent, and the solution is applied to the surface of the epidermis, whereby the applied solution can be allowed to penetrate into the epidermis. The applied solution permeates, for example, into the uppermost stratum corneum, further into the stratum granulosum below the stratum corneum, and further into the underlying layer. And the diacylglycerol PEG adduct amplifies the expression of transporter-related genes in epidermal cells in each layer within the epidermis into which the solution has penetrated.

As another method for allowing the diacylglycerol PEG adduct to reach the epidermis, the diacylglycerol PEG adduct can be allowed to reach the epidermis in a vesicle state. Such a vesicle is formed as a closed spherical shell consisting of a bilayer or multiple layers of the bilayers of the diacylglycerol PEG adduct, and hydrophilic PEG chains are arranged on the surface of the outermost layer. A vesicle of the diacylglycerol PEG adduct can be prepared and applied to the epidermal surface to penetrate into the epidermis. After the vesicle of the diacylglycerol PEG adduct reaches the epidermis, the vesicle is decomposed and separated into individual molecules, whereby the diacylglycerol PEG adduct itself can exert its action.

In the conventional drug delivery system, the diacylglycerol PEG adduct, which is a vesicle material, has been considered as a mere carrier of the target substance. However, in the present invention, the diacylglycerol PEG adduct itself is utilized as an active ingredient. Therefore, in the present invention, the target substance incorporated into the vesicle in the ordinary drug delivery system is basically unnecessary. In the present invention, the diacylglycerol PEG adduct itself can function as an expression amplifier of transporter-related genes in epidermal cells by allowing the vesicle formed by mixing only water and the diacylglycerol PEG adduct to penetrate into the epidermis.

Some diacylglycerol PEG adducts spontaneously form vesicles when mixed with water at a predetermined temperature (see Japanese Patent No. 4497765 and No. 6297737). For example, by mixing and stirring 2% by mass of GDM12 or GDO12 with 98% by mass of deionized water at room temperature, a suspension of vesicles of GDM12 or GDO12 is obtained. As another example, a suspension of vesicles of GDS12 or GDS23 can be obtained by dissolving 2% by mass of GDS12 or GDS23 at 45 to 55° C., and then mixing and stirring with 98% by mass of deionized water at 45 to 55° C. As yet another example, a suspension of vesicles of GDP23 can be obtained by dissolving 2% by mass of GDP23 at 37° C. and then mixing and stirring with 98% by mass of deionized water at 37° C. The vesicles remain stable even when the suspension obtained at a temperature higher than room temperature is cooled to room temperature.

As another example, the use of a vesicle formed by mixing an aqueous solution of various substances in place of the water described above with a diacylglycerol PEG adduct and stirring the mixed solution is also included in the scope of the present invention. In this case, the substance contained in the aqueous solution may have another function.

As still another example, a case where the surface of a vesicle formed by mixing and stirring water or an aqueous solution and a diacylglycerol PEG adduct is modified with a charged element such as a cationic surfactant is also included in the scope of the present invention. Japanese Patent No. 6297737 describes that a positively charged vesicle is particularly excellent in permeability into the epidermis and retention.

Hereinafter, the relation between the application of diacylglycerol PEG adducts to epidermal cells and transporter-related genes in epidermal cells will be shown by test data.

### (1) Test for Amplification of Expression of Transporter-Related Genes in Epidermal Cells

A test for confirming the amplification of mRNA expression of each transporter-related gene in epidermal cell was performed.

### (1-1) Test Method

Normal human epidermal keratinocytes (NHEKs) were seeded on a 96-well plate at a cell density of 2.0×10⁴cells/well using HuMedia-KG2 medium (manufactured by Kurabo Industries Ltd.), and cultured under the conditions of 37° C. and 5% CO₂ for 24 hours.

Thereafter, a diacylglycerol PEG adduct was added thereto using HuMedia-KB2 medium (manufactured by Kurabo Industries Ltd.), and each was cultured under the conditions of 37° C. and 5% CO₂ for a predetermined time. Table 1 represents the target gene, the types of diacylglycerol PEG adduct added, the adding amount, and the culture time. A control (no addition) was also cultured under the same conditions.

TABLE 1

| Target gene | Type | Amount(μM) | Culture time(hr) |
|---|---|---|---|
| SVCT2 | GDS23 | 50 | 24, 30, 36 |
| | GDM12 | 100 | 30 |
| SLC7A11 | GDS23 | 50 | 18, 24, 30, 36 |
| | GDM12 | 100 | 24 |
| GSR | GDS23 | 50 | 24 |
| | GDM12 | 100 | 30 |
| ABCC1 | GDS23 | 50 | 24 |
| | GDM12 | 100 | 30 |
| AQP3 | GDS23 | 50 | 18, 24, 30, 36 |
| | GDM12 | 100 | 24, 30 |

After the cells were cultured for each predetermined time, RNA was extracted from the cells of each sample and control. The extracted RNA was reverse-transcribed to prepare cDNA, and the mRNA of the target gene was quantified by quantitative real-time PCR expression analysis. GAPDH (glyceraldehyde 3-phosphate dehydrogenase) was used as an internal standard.

In the analysis, the mRNA expression level of each target gene was corrected with the value of the expression level of GAPDH, which is an internal standard in the same sample, and the correction value of the sample was calculated as a relative expression level when the correction value of the control was set to 1.

### (1-2) Test Results

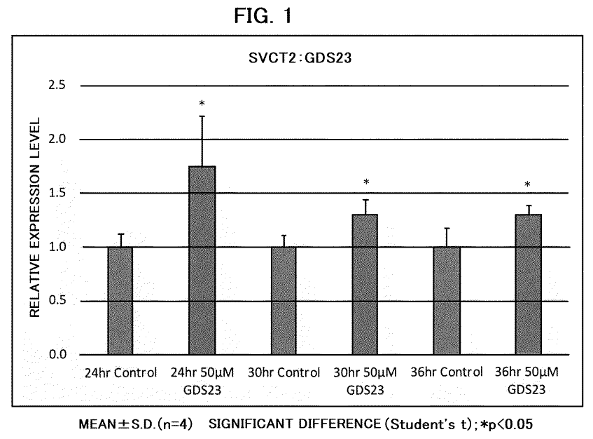

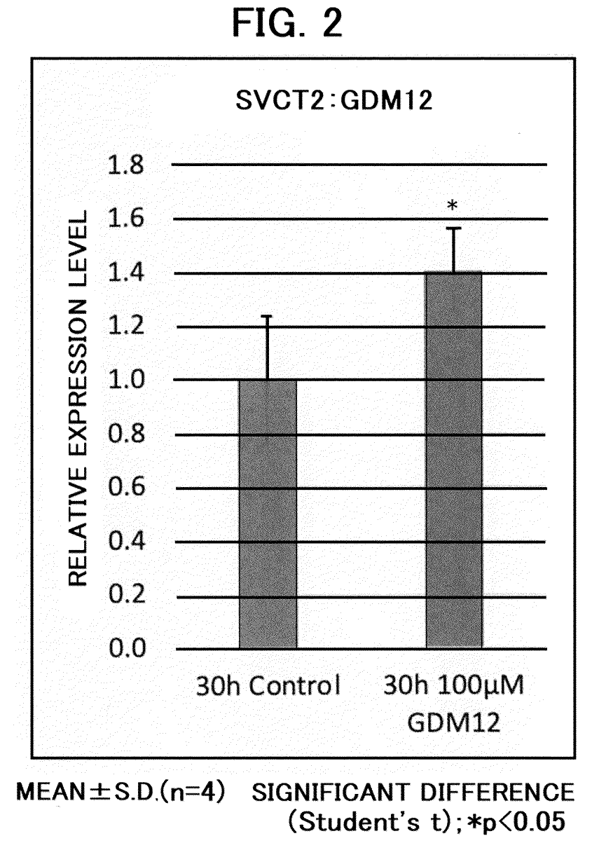

FIG. 1 and FIG. 2 are graphs illustrating the relative expression levels of SVCT2 genes in GDS23 and GDM12, respectively. The expression level in GDS23 was confirmed to be about 1.3 to 1.7 times that of the control, and the expression level in GDM12 was confirmed to be about 1.4 times that of the control.

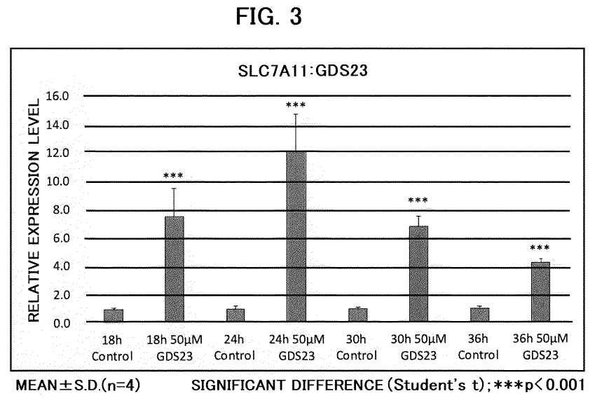

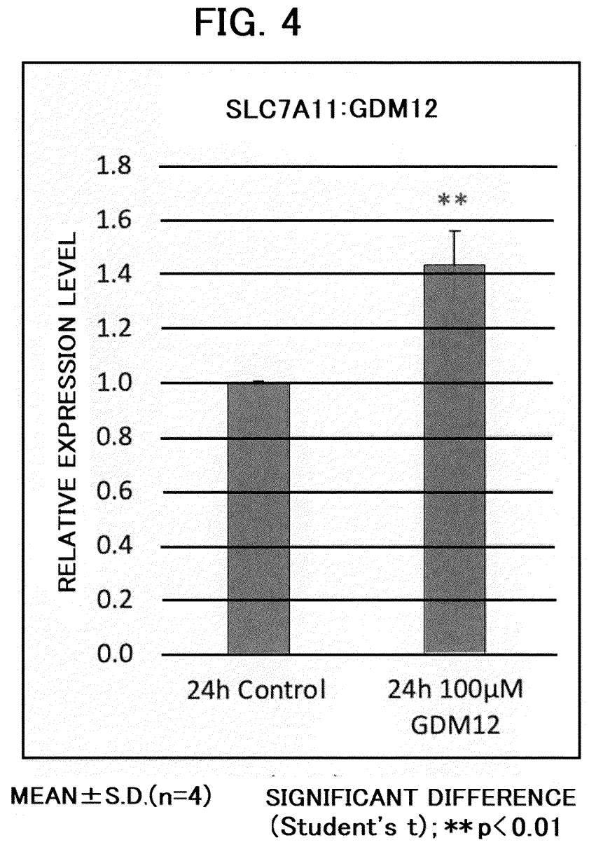

FIG. 3 and FIG. 4 are graphs illustrating the relative expression levels of the SLC7A11 genes in GDS23 and GDM12, respectively. The expression level in GDS23 was confirmed to be about 4 to 12 times that of the control, and the expression level in GDM12 was confirmed to be about 1.4 times that of the control.

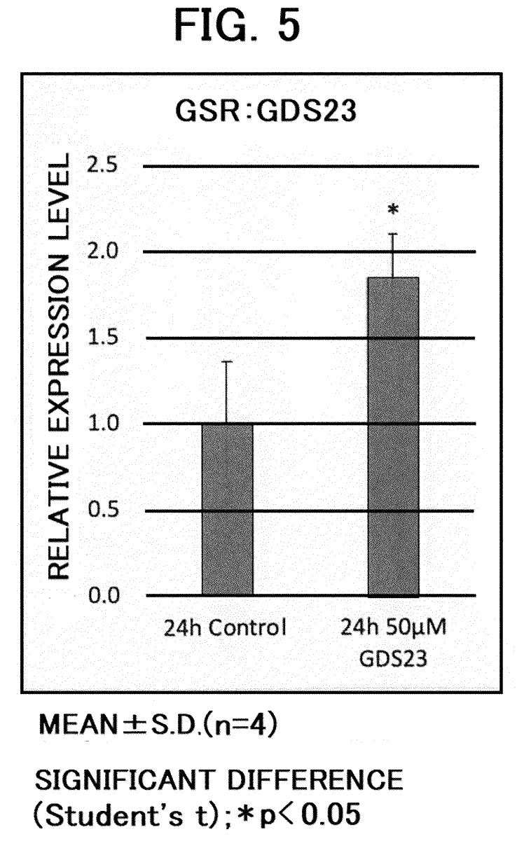

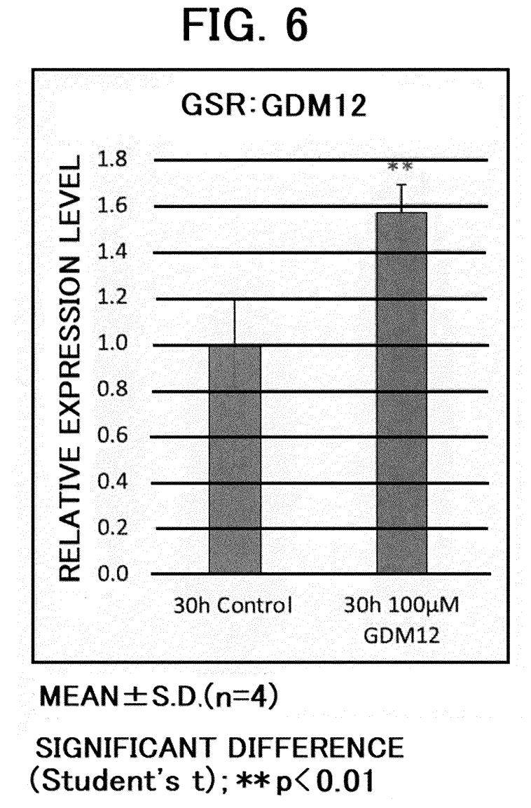

FIG. 5 and FIG. 6 are graphs illustrating the relative expression levels of GSR genes in GDS23 and GDM12, respectively. The expression level in GDS23 was confirmed to be about 1.8 times that of the control. The expression level in GDM12 was confirmed to be about 1.5 times that of the control.

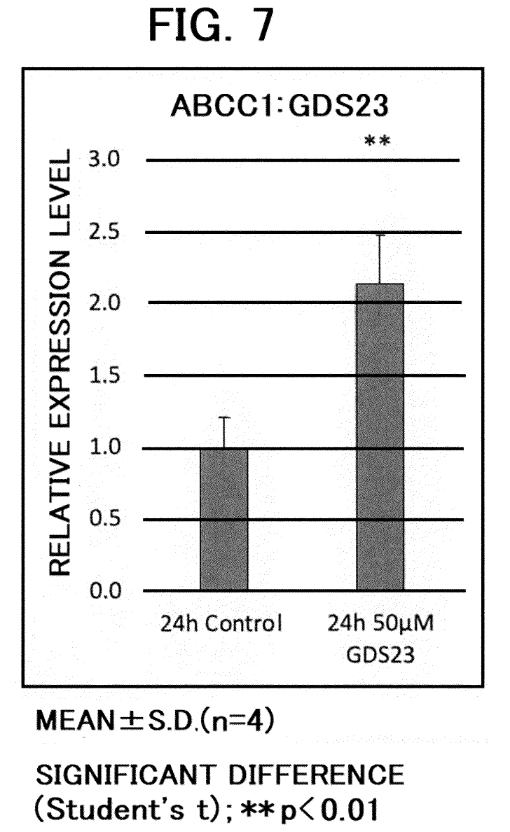

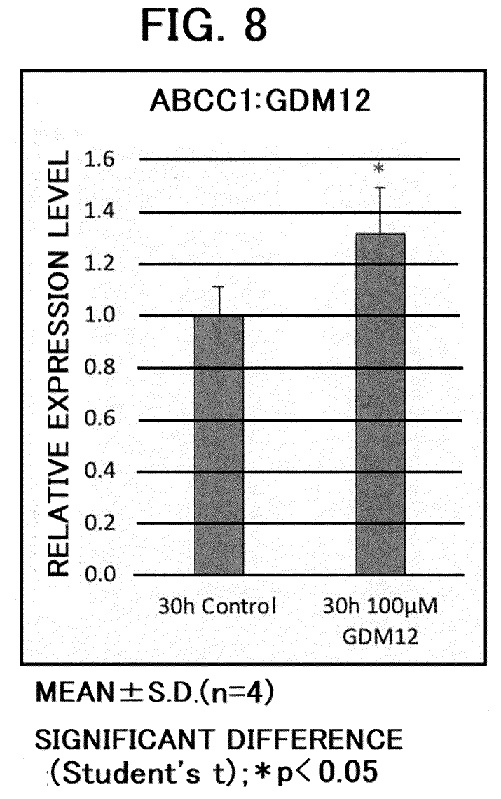

FIG. 7 and FIG. 8 are graphs illustrating the relative expression levels of ABCC1 genes in GDS23 and GDM12, respectively. The expression level in GDS23 was confirmed to be about 2.1 times that of the control. The expression level in GDM12 was confirmed to be about 1.3 times that of the control.

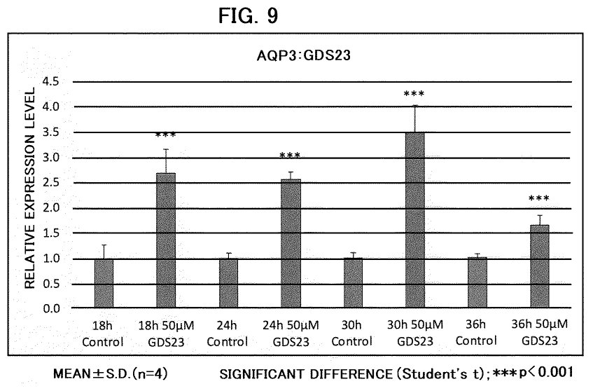

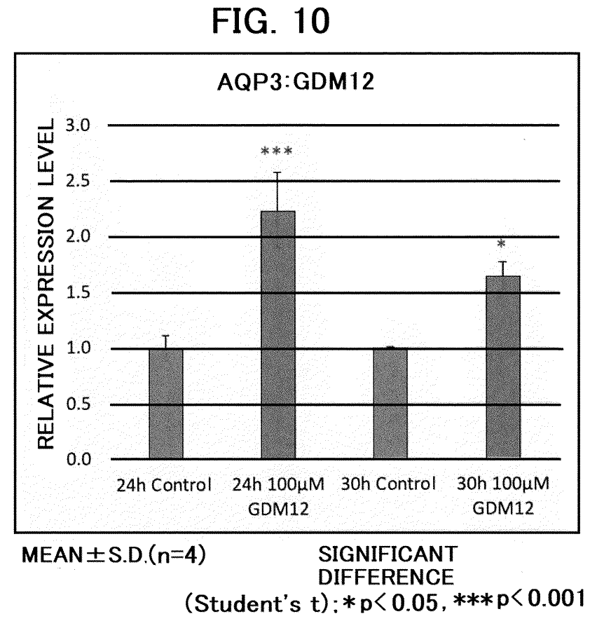

FIG. 9 and FIG. 10 are graphs illustrating the relative expression levels of aquaporin 3 genes in GDS23 and GDM12, respectively. The expression level in GDS23 was confirmed to be about 1.6 to 3.5 times that of the control. The expression level in GDM12 was confirmed to be about 1.6 to 2.2 times that of the control.

### (2) Test for Enhanced Production of SVCT2 Proteins

A test for confirming the enhanced production of proteins encoded by the SVCT2 genes was performed using Western blotting.

### (2-1) Test Method

Normal human epidermal keratinocytes (NHEKs) were seeded on a 96-well plate at a cell density of 1.0×10⁵ cells/well using HuMedia-KG2 medium and cultured under the conditions of 37° C. and 5% CO₂ for 24 hours.

Thereafter, 50 μM of PEG-23 glycerol distearate (GDS23) was added thereto using HuMedia-KB2 medium, and the cells were cultured under the conditions of 37° C. and 5% CO₂ for 24 hours. A control (no addition) was cultured only in HuMedia-KB2 medium.

After culturing, Western blotting was applied. First, proteins were extracted from the cells using SDS-PAGE containing 10% mercaptoethanol. The protein extract solution was subjected to electrophoresis using a polyacrylamide gel, and the proteins were transferred to a membrane by a semi-dry method. The SVCT2 protein was quantified using predetermined primary antibodies and secondary antibodies shown in Table 2. The amount of the SVCT2 protein was corrected with the amount of GAPDH, which is an internal standard in the same sample, and the correction value of the sample was calculated with the correction value of the control set to 1.

TABLE 2

| Target | | Dilution | Derived | Secondary | Dilution |
|---|---|---|---|---|---|
| protein | Primary antibody | rate | species | antibody | rate |
| GAPDH | Anti-GAPDH | ×1/10,000 | Mouse | Rabbit Anti-Mouse | ×1/10,000 |
| | antibody [6C5] - | | monoclonal | IgG H&L (HRP) | |
| | Loading Control | | | ab6728 (Abcam PLC) | |
| | ab8245 (Abcam PLC) | | | | |
| SVCT2 | SVCT2 Polyclonal | ×1/3,000 | Rabbit | Goat Anti-Rabbit | ×1/10,000 |
| | antibody (Proteintech | | Polyclonal | IgG H&L (HRP) | |
| | Group, Inc.) | | | ab205718 | |
| | | | | (Abcam PLC) | |

### (2-2) Test Results

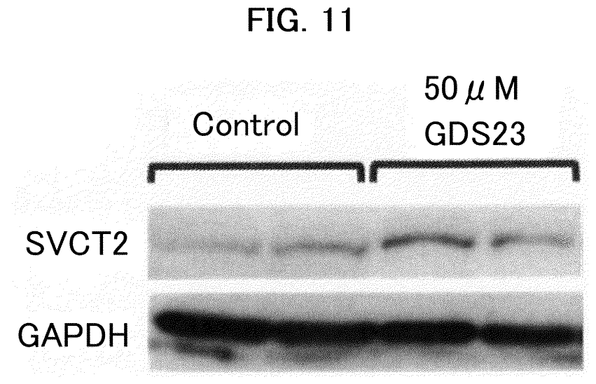

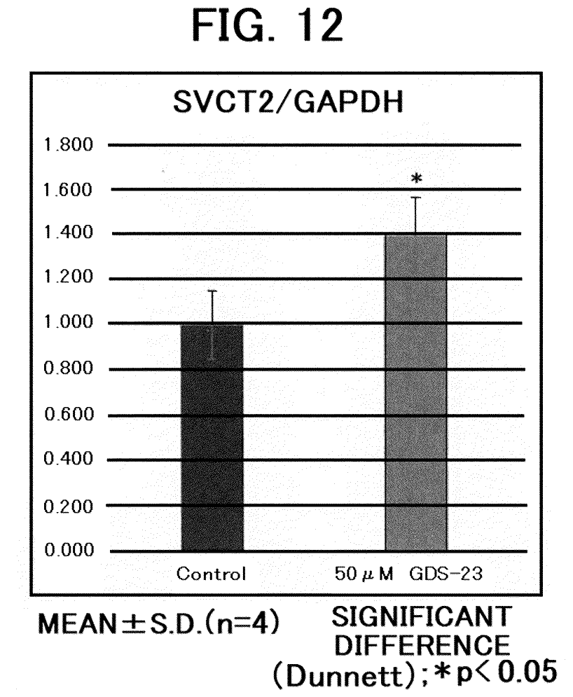

FIG. 11 is a band image of Western blot of the SVCT2 protein with respect to GDS23. FIG. 12 is a graph illustrating the expression level of the SVCT2 protein.

As illustrated in FIG. 11, an increase in the SVCT2 protein was confirmed in the GDS23 added sample. As illustrated in FIG. 12, the amount of the SVCT2 protein in the GDS23 added sample was confirmed to be about 1.4 times that in the control sample.

### (3) Evaluation Test of Cellular Uptake Action of Vitamin C (Ascorbic Acid) by Increasing SVCT2

From the test results of (1) and (2) described above, it was confirmed that the expression of the SVCT2 genes was enhanced by the application of the diacylglycerol PEG adduct to epidermal cells, and thus the production of the protein, which is the vitamin C transporter encoded thereby, was enhanced. Therefore, the cellular uptake action of vitamin C was actually evaluated. This evaluation was performed by quantifying the cytotoxicity of hydroquinone (HQ) when a diacylglycerol PEG adduct, vitamin C, and hydroquinone were added to epidermal cells.

Hydroquinone itself has no cytotoxicity and has a whitening action, but exhibits cytotoxicity when oxidized to benzoquinone (see Japanese Patent No. 6860739). On the other hand, when hydroquinone is added to the epidermis together with an oxidation inhibitor, hydroquinone is not oxidized and cytotoxicity is supposed to be able to be inhibited (the same applies to the following evaluation test (4)).

### (3-1) Test Method

Normal human epidermal keratinocytes (NHEKs) were seeded on a 96-well plate at a cell density of 2.0×10⁴ cells/well using HuMedia-KG2 medium and cultured under the conditions of 37° C. and 5% CO: for 24 hours.

Thereafter, using HuMedia-KB2 medium, a sample to which 50 μM of PEG-23 glycerol distearate (GDS23) was added and a sample to which GDS23 was not added were respectively cultured under the conditions of 37° C. and 5% CO: for 24 hours.

Subsequently, the samples were washed with PBS (−), and the GDS23-added samples and the GDS23 non-added samples were respectively cultured in the following six media for 24 hours.
HuMedia-KB2 medium containing 0 μM of ascorbic acid (AsA)
HuMedia-KB2 medium containing 1 μM of ascorbic acid (AsA)
HuMedia-KB2 medium containing 5 μM of ascorbic acid (AsA)
HuMedia-KB2 medium containing 10 μM of ascorbic acid (AsA)
HuMedia-KB2 medium containing 50 μM of ascorbic acid (AsA)
HuMedia-KB2 medium containing 100 μM of ascorbic acid (AsA)

Further, the cells were washed with PBS (−) and cultured in HuMedia-KB2 medium containing 400 μM of hydroquinone (HQ) for 24 hours.

The control (no addition of any of GDS23, AsA, and HQ) was cultured only in HuMedia-KB2 medium.

Thereafter, the cell viability was measured by the neutral red assay. The cell viability was calculated for each of the GDS23-added samples and the GDS23 non-added samples, taking the cell viability of the control as 100%.

### (3-2) Test Results

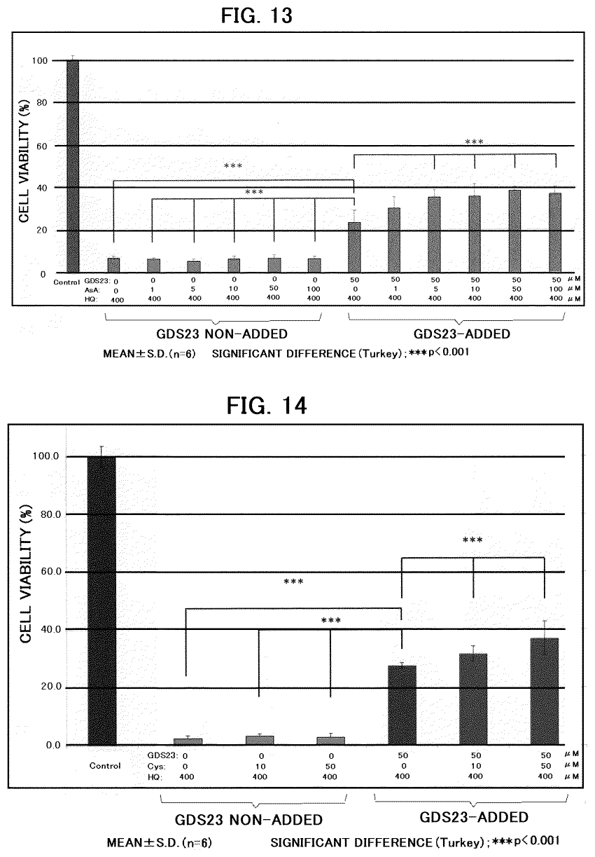

FIG. 13 is a graph illustrating the evaluation test results of the cellular uptake action of vitamin C with respect to GDS23. Except for the control at the leftmost end, the left six samples are GDS23 non-added samples, and the right six samples are GDS23-added samples.

Among the six GDS23 non-added samples, the leftmost sample is an ascorbic acid non-added sample, and the other five samples are ascorbic acid-added samples. When these samples are compared, it is found that the cell viability of the ascorbic acid-added samples at a low concentration of 5 to 100 μM are not different from that of the ascorbic acid non-added sample, and that ascorbic acid has almost no effect on the cytotoxicity caused by hydroquinone.

On the other hand, the cell viability of the GDS23-added samples is higher than that of the GDS23 non-added sample, and the cytotoxicity by hydroquinone is significantly reduced. Among the six GDS23-added samples, the cell viability of the ascorbic acid-added samples is higher than that of the ascorbic acid non-added sample at the leftmost end. This is considered to be due to the increase of SVCT2 by GDS23, which increased the uptake of ascorbic acid into the cells, increased the intracellular concentration of ascorbic acid, and inhibited the oxidation of hydroquinone.

It is considered that the reason why the cell viability is increased even in the ascorbic acid non-added sample among the GDS23-added samples as compared with the GDS23 non-added sample is due to the HQ cytotoxicity reducing action of GDS23 itself disclosed in Japanese Patent No. 6860739, and is considered to be due to a mechanism different from the vitamin C transporter-increasing action of the present invention.

### (4) Evaluation Test of Cellular Uptake Action of Cystine by Increase in SLC7A11

From the test results of (1) and (2) described above, it was confirmed that the expression of the SLC7A11 genes was enhanced by the application of the diacylglycerol PEG adduct to epidermal cells, and thus the production of the protein, which is the xCT transporter encoded thereby, was enhanced. Therefore, the cellular uptake action of cystine was actually evaluated. This evaluation was performed by quantifying the cytotoxicity of hydroquinone (HQ) when a diacylglycerol PEG adduct, cystine, and hydroquinone were added to epidermal cells.

### (4-1) Test Method

Normal human epidermal keratinocytes (NHEKs) were seeded on a 96-well plate at a cell density of 2.0×10⁴ cells/well using HuMedia-KG2 medium and cultured under the conditions of 37° C. and 5% CO₂ for 24 hours.

Thereafter, using HuMedia-KB2 medium, a sample to which 50 μM of PEG-23 glycerol distearate (GDS23) was added and a sample to which GDS23 was not added were respectively cultured under the conditions of 37° C. and 5% CO₂ for 24 hours.

Subsequently, the samples were washed with PBS (−), and the GDS23-added samples and the GDS23 non-added samples were respectively cultured in the following three media for 30 hours. The cysteine added to the medium is oxidized in the medium or between cells to be converted into cystine, which is a dimer, and is incorporated into the cells as cystine.
HuMedia-KB2 medium containing 0 μM of cysteine (Cys)
HuMedia-KB2 medium containing 10 μM of cysteine (Cys)
HuMedia-KB2 medium containing 50 μM of cysteine (Cys)

Further, the cells were washed with PBS (−) and cultured in HuMedia-KB2 medium containing 400 μM of hydroquinone (HQ) for 24 hours.

The control (no addition of any of GDS23, Cys, and HQ) was cultured only in HuMedia-KB2 medium.

Thereafter, the cell viability was measured by the neutral red assay. The cell viability was calculated for each of the GDS23-added samples and the GDS23 non-added samples, taking the cell viability of the control as 100%.

### (4-2) Test Results

FIG. 14 is a graph illustrating the evaluation test results of the cellular uptake action of cystine with respect to GDS23. Except for the control at the leftmost end, the left three samples are GDS23 non-added samples, and the right three samples are GDS23-added samples.

Among the three GDS23 non-added samples, the leftmost sample is a cysteine non-added sample, and the other two samples are cysteine-added samples. When these samples are compared, it is found that the cell viability of the 10 μM and 50 μM of cysteine-added samples are not different from that of the cysteine non-added sample, and that cysteine (cystine) has almost no effect on the cytotoxicity caused by hydroquinone. That is, it is found that the uptake of cystine into the cells is not performed in GDS23 no-added samples.

On the other hand, the cell viability of the GDS23-added samples is higher than that of the GDS23 non-added sample, and the cytotoxicity by hydroquinone is significantly reduced. Among the three GDS23-added samples, the cell viability of the other two cysteine-added samples is higher than that of the cysteine non-added sample at the leftmost end. This is considered to be due to the increase of SLC7A11 by GDS23, which increased the uptake of cystine into the cells, increased the intracellular concentration of cystine, enhanced the production of glutathione, and inhibited the oxidation of hydroquinone.

It is considered that the reason why the cell viability is increased even in the cysteine non-added sample on the leftmost side among the GDS23-added samples as compared with the GDS23 non-added sample is due to the HQ cytotoxicity reducing action of GDS23 itself disclosed in Japanese Patent No. 6860739, and is considered to be due to a mechanism other than the x-CT transporter increasing action of the present invention.

### (5) Preparation of Cosmetic and Skin External Preparation

In the following descriptions, preparation examples of a cosmetic and a skin external preparation containing an expression amplifier for transporter-related genes in epidermal cells containing a diacylglycerol PEG adduct as an active ingredient are shown. Each of the following preparation examples contains an ascorbic acid derivative. The ascorbic acid derivative has better stability and skin permeability as compared to ascorbic acid. The ascorbic acid derivative acts by being converted into ascorbic acid by decomposition or enzymatic action after penetration into the skin, or acts in the same manner as ascorbic acid as it is. Examples of the ascorbic acid derivative include 3-O-ethyl ascorbic acid, 3-lauryl glyceryl ascorbate, myristyl 3-glyceryl ascorbate, and the like. The cosmetic or the like of the present invention can contain either or both of ascorbic acid and an ascorbic acid derivative.

### (5-1) Preparation Example of Lotion

The lotion of preparation example 1 contains PEG-12 glycerol dimyristate, PEG-23 glycerol distearate, ascorbic acid and derivatives thereof, and glutathione.

TABLE 3
Preparation example 1 (lotion)

| Ingredients | Mass (%) |
|---|---|
| Water | 68.40% |
| Glycerin | 12.00% |
| Propanediol | 9.00% |
| PEG-12 glycerol dimyristate | 3.00% |
| PEG-23 glycerol distearate | 3.00% |
| Squalane | 1.50% |
| Ascorbic acid | 1.00% |
| 3-O-ethyl ascorbic acid | 0.50% |
| 3-lauryl glyceryl ascorbate | 0.10% |
| Glutathione | 0.01% |
| Allantoin | 0.10% |
| Cholesterol | 0.10% |
| Tocopherol | 0.01% |
| Lactic acid | 0.20% |
| Stearamide propyl dimethylamine | 0.50% |
| Phenoxyethanol | 0.50% |
| EDTA-2Na | 0.08% |

### (5-2) Preparation Example of Milky Lotion

The milky lotion of preparation example 2 contains PEG-12 glycerol dimyristate, PEG-23 glycerol distearate, ascorbic acid and derivatives thereof, cysteine, and glutathione.

TABLE 4
Preparation example 2 (milky lotion)

| Ingredients | Mass (%) |
|---|---|
| Water | 61.00% |
| Glycerin | 8.00% |
| Squalane | 8.00% |
| BG | 6.00% |
| PEG-12 glycerol dimyristate | 10.00% |
| PEG-23 glycerol distearate | 0.50% |
| Ascorbic acid | 0.50% |
| 3-O-ethyl ascorbic acid | 0.50% |
| Cysteine | 0.10% |
| Glutathione | 0.05% |
| Niacinamide | 1.00% |
| Cholesteryl oleate | 1.50% |
| Cholesteryl stearate | 0.50% |
| Cholesterol | 0.05% |
| Tocopherol | 0.05% |
| Rosa Canina Fruit Oil | 0.02% |
| Arginine | 0.20% |
| Ethylhexylglycerin | 0.30% |
| (Dimethicone/vinyl dimethicone) crosspolymer | 0.65% |
| Carbomer | 0.50% |
| Phenoxyethanol | 0.50% |
| EDTA-2Na | 0.08% |

### (5-3) Preparation Example of Aqueous Gel

The aqueous gel of preparation example 3 contains PEG-12 glycerol dimyristate, PEG-23 glycerol distearate, ascorbic acid and derivatives thereof, and glutathione.

TABLE 5
Preparation example 3 (aqueous gel)

| Ingredients | Mass (%) |
|---|---|
| Water | 74.00% |
| PEG-23 glycerol distearate | 10.00% |
| Diglycerin | 4.00% |
| Glycerin | 4.00% |
| Pentyleneglycol | 2.00% |
| Cyclohexylglycerin | 1.00% |
| Glutathione | 1.00% |
| PEG-12 glycerol dimyristate | 0.50% |
| Ascorbic acid | 0.50% |
| Myristyl 3-glyceryl ascorbate | 0.10% |
| Niacinamide | 1.00% |
| (PEG-240/ decyl tetradeceth-20/HDI) copolymer | 1.00% |
| Allantoin | 0.10% |
| Panthenol | 0.10% |
| Citric acid | 0.05% |
| Sodium citrate | 0.05% |
| Lavender oil | 0.20% |
| Phenoxyethanol | 0.40% |

### (5-4) Preparation Example of Hydroquinone-Containing Cream

The hydroquinone-containing cream of preparation example 4 contains PEG-12 glycerol dimyristate, PEG-23 glycerol distearate, ascorbic acid and derivatives thereof, and glutathione in addition to hydroquinone. The cytotoxicity of hydroquinone is inhibited by the antioxidant action of these substances. As a result, the cosmetic effect of hydroquinone is exhibited.

TABLE 6
Preparation example 4 (hydroquinone-containing cream)

| Ingredients | Mass(%) |
|---|---|
| Water | 44.75% |
| Propanediol | 10.00% |
| BG | 10.00% |
| Squalane | 10.00% |
| PEG-23 glycerol distearate | 5.00% |
| Hydroquinone | 4.00% |
| Pentaerythrityl tetraethylhexanoate | 2.00% |
| PEG-12 glycerol dimyristate | 1.00% |
| Ascorbic acid | 0.50% |
| Glutathione | 0.50% |
| Hexyl 3-glyceryl ascorbate | 0.20% |
| 3-O-ethyl ascorbic acid | 0.10% |
| Cysteine | 0.10% |
| Cholesteryl macadamiate | 2.00% |
| Behenyl alcohol | 2.00% |
| Stearyl alcohol | 2.00% |
| Dimethicone | 1.80% |
| Glyceryl stearate | 1.50% |
| Tocopherol acetate | 0.10% |
| Sodium stearoylmethyltaurine | 0.80% |
| Cholesteryl stearate | 0.10% |
| Polyglyceryl stearate-10 | 0.80% |
| Potassium lactate | 0.04% |
| Lactic acid | 0.20% |
| Linalool | 0.08% |
| Bitter orange flower oil | 0.04% |
| Phenoxyethanol | 0.10% |
| EDTA-2Na | 0.10% |
| Sodium pyrosulfite | 0.19% |

### (5-5) Preparation Example of Hydroquinone-Containing Ointment

The hydroquinone-containing ointment of preparation example 5 contains PEG-23 glycerol distearate, ascorbic acid, glutathione, and cysteine in addition to hydroquinone.

TABLE 7
Preparation example 5 (hydroquinone-containing ointment)

| Ingredients | Mass (%) |
|---|---|
| Propanediol | 23.00% |
| PEG-23 glycerol distearate | 12.00% |
| Ascorbic acid | 12.00% |
| Water | 12.00% |
| Hydroquinone | 10.00% |
| BG | 10.00% |
| Stearyl alcohol | 9.00% |
| Dimethicone | 6.00% |
| Trimethylolpropane triethylhexanoate | 3.00% |
| PEG-12 glycerol dimyristate | 1.00% |
| Glutathione | 0.25% |
| Cysteine | 0.10% |
| Dimethicone crosspolymer | 0.45% |
| (Dimethicone/vinyl dimethicone) crosspolymer | 0.40% |
| Lactic acid | 0.40% |
| Pelargonium graveolens oil | 0.10% |
| Phenoxyethanol | 0.10% |
| EDTA-2Na | 0.10% |
| Sodium pyrosulfite | 0.10% |

Although not exemplified, there are various combinations of ingredients other than the diacylglycerol PEG adduct in addition to those described above. One or more ingredients of the group consisting of ascorbic acid, an ascorbic acid derivative, glutathione, and cysteine are optionally selected. In that case, hydroquinone can be further contained. The same applies to skin external preparations other than cosmetics.

Although the present invention has been described with reference to the embodiments, the present invention is not limited to these embodiments, and modifications obvious from these embodiments are also included in the present invention.

The invention claimed is:

1. A method for amplifying expression of a transporter-related gene in epidermal cells, the method comprising applying a diacylglycerol PEG adduct having a following structural formula; 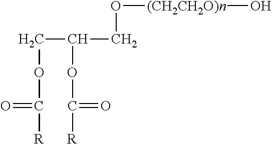
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

2. A method for using an active ingredient for amplifying expression of a transporter-related gene in epidermal cells in a cosmetic or a skin external preparation, the method comprising using, as the active ingredient, a diacylglycerol PEG adduct having a following structural formula; 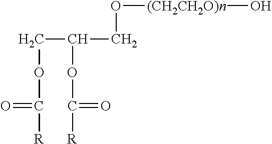
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

3. The method according to claim 1, wherein the transporter-related gene in epidermal cells is at least one of a group consisting of SVCT2, SLC7A11, glutathione reductase, ABCC1, and aquaporin 3.

4. The method according to claim 1, wherein the diacylglycerol PEG adduct permeates into epidermis in a solution state or a vesicle state.

5. The method according to claim 2, wherein the cosmetic or the skin external preparation further comprises at least one of a group consisting of ascorbic acid, an ascorbic acid derivative, glutathione, and cysteine.

6. The method according to claim 5, further comprising hydroquinone.
